(19) United States
(12) Patent Application Publication
(10) Pub. No.: US 2026/0345133 A1
(43) Pub. Date: Nov. 5, 2026

(54) AUTOMATIC MID-STREAM URINE COLLECTION DEVICE

(71) Applicant: IMAM ABDULRAHMAN BIN FAISAL UNIVERSITY, Dammam (SA)

(72) Inventors: Moaaz Abdulrazack A. AMIR, Dammam (SA); Baraa Abdulrazack A. AMIR, Dammam (SA); Amaar Abdulrazack A. AMIR, Dammam (SA)

(73) Assignee: IMAM ABDULRAHMAN BIN FAISAL UNIVERSITY, Dammam (SA)

(21) Appl. No.: 19/199,122

(22) Filed: May 5, 2025

(51) Int. Cl. A61B 10/00

(52) U.S. Cl. CPC A61B 10/007

(57) ABSTRACT

A sample collection device and a method for the collection of a urine sample includes a main chassis with a funnel; a forestream isolation tube having an opening at a top portion; a collection cup with a bottom cap containing a sponge holder having a sponge therein; and a plunger having a top end inserted in a bottom portion of the forestream isolation tube and a bottom end extending toward the sponge. The sample collection device is configured such that urine directed into the funnel flows through the funnel into the main chassis and through the opening of the forestream isolation tube to the sponge. When the urine reaches the sponge, the sponge absorbs the urine and expands, pushing the plunger upwards to seal the opening of the forestream isolation tube, separating forestream from midstream portions.

### BACKGROUND

### Technical Field

The present disclosure relates generally to medical sample collection devices, and more specifically, to a device for the automated collection of a mid-stream urine sample.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Traditionally, urine samples are routinely collected for many patients and can provide information on the chemical profile of the urine as well as the presence or absence of sediment, cells, and microorganisms. Multiple methods exist for collection including first void collection, mid-stream collection, and collection via a catheter or suprapubic aspiration. The universal gold standard for collection is the mid-stream catch, which involves instructing the patient to initially urinate into the toilet for the first few seconds, then to continue urinating into the collection cup. However, several challenges exist. Above all, because of the messy and rather crude method of collection, patients typically suffer from some degree of noncompliance. This can lead to contaminated urine samples which can hamper diagnosis and treatment of said patients, and potentially lead to increase in antibiotic resistant organisms, which in turn may further complicate patient care.

The impact of non-compliance with proper mid-stream collection can be seen on both the healthcare system and the patients directly. Regarding the healthcare systems, contaminated samples lead to repeat tests which increase time and financial resources. Furthermore, due to the high contamination rates, there is unnecessary prescription of antibiotics, which increases the rates of antibiotic resistant infections that represent a multibillion-dollar burden on healthcare systems worldwide. Regarding the impact on the end user/patient, the current collection methods create an unpleasant urine collection experience as it requires manual control and is less hygienic. Additionally, inaccurate collection leads to less accurate diagnoses and subsequent treatment, ultimately compromising patient care. This also exposes patients to unnecessary antibiotics with possible drug side effects.

Currently, alternative solutions for streamlining the collection process of mid-steam urine exist but have significant limitations. Some alternatives involve large containers that are connected to the toilet seat directly, which is rather unpractical and difficult to manage by the hospital staff and requires large storage space. Other products rely on purging excess urine outside of the container and into the toilet, meaning that collection is only possible if the patient is on the toilet, and not possible in places like the bed. It is worth noting that most existing products are proprietary and compete against the current hardware used in the hospitals, rather than working in conjunction with currently used technology. Some of the known devices/methods are discussed in the proceeding paragraphs.

U.S. Pat. No. 4,494,581A describes a device for isolating forestream and midstream portions of collected urine samples using a valve controlled by a material that swells when exposed to urine. The device includes a forestream receiving chamber with a normally open inlet aperture toward which urine is directed by a funnel. Upon contact with liquid, the material in the chamber swells and forcefully urges a movable member against the aperture to block further entry of liquid, redirecting subsequently received liquid into a midstream specimen collection container. However, this reference does not disclose an integrated sample collection device with an automatic mechanism for separating forestream and midstream urine that maintains a fully enclosed collection environment throughout the collection process having a top end inserted in a bottom portion of the forestream isolation tube with a base having a larger diameter than the cylinder portion.

U.S. Pat. No. 4,492,258A describes a sterile urine specimen collection device that collects the initial forestream portion of a void in a chamber and directs the midstream overflow into a specimen container. The device includes a forestream collection chamber containing a urine-absorbent solid body that immobilizes the collected forestream portion, preventing it from intermixing with the subsequently received midstream portion. However, this reference does not disclose an integrated sample collection device with an automatic mechanism for separating forestream and midstream urine that maintains a fully enclosed collection environment throughout the collection process having a top end inserted in a bottom portion of the forestream isolation tube with a base having a larger diameter than the cylinder portion.

US20180168557A1 describes a specimen collection container having a separation chamber that includes a first chamber, a second chamber, and a valve located between them. The valve transitions from an open position that permits fluid communication between the chambers to a closed position that maintains fluid isolation, allowing a predetermined volume of fluid to pass from the first chamber to the second chamber before closing. However, this reference does not disclose an integrated sample collection device with an automatic mechanism for separating forestream and midstream urine that maintains a fully enclosed collection environment throughout the collection process having a top end inserted in a bottom portion of the forestream isolation tube with a base having a larger diameter than the cylinder portion.

US20040267159A1 describes a dual-chamber device for receiving and containing urine that employs a generally cylindrical body with a detachable top cover and an internal, transverse, funnel-shaped wall. The device includes a floatable orifice stopper in the lower chamber that responds to fore-stream liquid filling the chamber to a predetermined level by closing the orifice, causing subsequent mid-stream urine to be received into the upper chamber. However, this reference does not disclose an integrated sample collection device with an automatic mechanism for separating forestream and midstream urine that maintains a fully enclosed collection environment throughout the collection process having a top end inserted in a bottom portion of the forestream isolation tube with a base having a larger diameter than the cylinder portion.

WO2024033193A1 describes an apparatus for facilitating forestream collection of urine comprising an inlet funnel, an overflow outlet, a collection outlet connectable to a urine collection receptacle, and a member expandable on absorption of urine that is at least partially located in the collection outlet. The apparatus includes a flow-through section between the inlet funnel and outlets to allow urine to flow while minimizing contact with the expandable member. However, this reference does not disclose an integrated sample collection device with an automatic mechanism for separating forestream and midstream urine that maintains a fully enclosed collection environment throughout the collection process having a top end inserted in a bottom portion of the forestream isolation tube with a base having a larger diameter than the cylinder portion.

Peezy Urine collection device, as described in clinical trial registration ISRCTN68511881, is a funnel-shaped device that channels the early portion of the urine stream into a sponge and collects the midstream portion in a standard container. However, this reference does not disclose an integrated sample collection device with an automatic mechanism for separating forestream and midstream urine that maintains a fully enclosed collection environment throughout the collection process having a top end inserted in a bottom portion of the forestream isolation tube with a base having a larger diameter than the cylinder portion.

Each of the aforementioned references suffers from one or more drawbacks hindering their adoption, such as complex operation requiring patient training, inability to maintain complete separation between forestream and midstream portions, reliance on external pressure or flow rate for proper functioning, or incompatibility with standard collection containers used in medical settings. These existing devices require patients to manually redirect urine flow during collection, which decreases compliance and increases the risk of contamination. Other devices lack proper sealing mechanisms to prevent mixing of the forestream and midstream portions after collection. Additionally, some devices are designed as proprietary replacements for standard collection equipment, complicating integration into existing healthcare workflows. Accordingly, it is one object of the present disclosure to provide a sample collection device that addresses these limitations by offering a simple, one-step automated collection process that effectively isolates the contaminated forestream while collecting a clean midstream sample, all while maintaining compatibility with standard medical collection cups and caps.

### SUMMARY

In an aspect, a sample collection device for the collection of a urine sample is described, comprising a main chassis and a funnel therein, the funnel configured to couple with an inner surface of a top portion of the main chassis; a forestream isolation tube having at least one opening at a top portion of the forestream isolation tube; a collection cup having a top opening and a bottom cap therein, the bottom cap comprising a sponge holder; a sponge contained within the sponge holder; and a plunger having a top end inserted in a bottom portion of the forestream isolation tube and a bottom end extending downward proximal to and/or contacting the sponge, wherein the sample collection device is fluidly configured such that the urine sample directed into the funnel flows through an inner compartment of the funnel, into the main chassis, and through the opening of the top portion of the forestream isolation tube to the sponge, and wherein as the urine sample travels to the sponge, the sponge absorbs the urine sample and expands, pushing the base of the plunger upwards to seal the opening of the top portion of the forestream isolation tube.

In some embodiments, the main chassis has an open top.

In some embodiments, the forestream isolation tube has a capacity of at least 40 mL.

In some embodiments, the collection cup is configured to hold a clean sample of urine.

In some embodiments, the collection cup has a cap configured to couple with the funnel to close a mouth of the funnel.

In some embodiments, the forestream isolation tube has a diameter smaller than the diameter of the main chassis.

In some embodiments, the plunger has a cylinder portion.

In some embodiments, the plunger has a base having a diameter larger than a diameter of the cylinder portion.

In some embodiments, when the opening of the top portion of the forestream isolation tube is sealed, the urine sample enters the main chassis, fills the main chassis, and overflows through the open top of the main chassis into the collection cup.

In some embodiments, the sponge is a cellulose sponge.

In some embodiments, the collection cup threads onto an inferior aspect of the sample collection device.

In some embodiments, a midstream portion of the urine is diverted into the main chassis and collection cup when the cellulose sponge expands and closes the plunger.

In some embodiments, the cellulose sponge can expand to more than 10× of its original size.

In some embodiments, the sample collection device has a cap that is used to seal the sample collection device when it is attached to the urine collection cup.

In some embodiments, the cap is removed before collection of the urine sample.

In some embodiments, the components are an integrally-connected unit.

In some embodiments, the urine collection cup is fully enclosed.

In some embodiments, the sample collection components are fully enclosed unless the cap is removed.

In some embodiments, both the forestream portion and the midstream portion remain enclosed within the sample collection device during the urine collection process.

In another aspect, a method for collecting the midstream portion of the urine sample is described, comprising collecting a urine sample into the main chassis of the urine collection device; collecting the forestream portion of the urine sample within the forestream isolation tube; expanding the cellulose sponge when the forestream portion of the urine sample is collected in the forestream isolation tube; pushing the moveable plunger upwards to seal off the forestream isolation tube from the main chassis; catching the remaining midstream portion of the urine sample; diverting the midstream portion of the urine sample into the urine collection cup; and screwing on the collection cup cap to the collection cup.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is an exemplary superolateral view of a sample collection device for the collection of a urine sample, according to certain embodiments.

FIG. 1B is an exemplary lateral view of the sample collection device, according to certain embodiments.

FIG. 1C is an exemplary superior view of the sample collection device, according to certain embodiments.

FIG. 1D is an exemplary inferior view of the sample collection device with urine flow, according to certain embodiments.

FIG. 1E is an exemplary exploded view of the sample collection device showing forestream collection, according to certain embodiments.

FIG. 1F is an exemplary cross-sectional view of the sample collection device showing midstream collection, according to certain embodiments.

FIG. 2A is a side view of the plunger showing its movement before expansion of sponge therein, according to certain embodiments.

FIG. 2B is a cross-sectional view of a plunger of the sample collection device showing its movement before expansion of sponge therein, according to certain embodiments.

FIG. 3A is a side view of the plunger showing its movement after expansion of sponge therein, according to certain embodiments.

FIG. 3B is a cross-sectional view of the plunger showing its movement after expansion of sponge therein, according to certain embodiments.

FIG. 4 is an exemplary flowchart listing steps involved in a method for collecting the midstream portion of the urine sample, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

Aspects of this disclosure are directed to a sample collection device for the collection of a urine sample and a method for collecting the midstream portion of the urine sample. The present disclosure provides a solution for automated midstream urine collection that simplifies the collection process while improving sample quality. The sample collection device of the present disclosure operates through an automatic mechanism that separates the initial forestream portion of urine, which typically contains contaminants, from the subsequent midstream portion that is more representative of conditions within the bladder. This separation occurs without requiring manual intervention from the patient, reducing the variability and potential for contamination associated with traditional collection methods. The sample collection device is adapted to work with existing medical collection containers, facilitating integration into current healthcare workflows without disrupting established supply chains or requiring proprietary components. The sample collection device aims to increase patient compliance with proper collection techniques, potentially reducing rates of sample contamination and subsequent unnecessary antibiotic prescriptions.

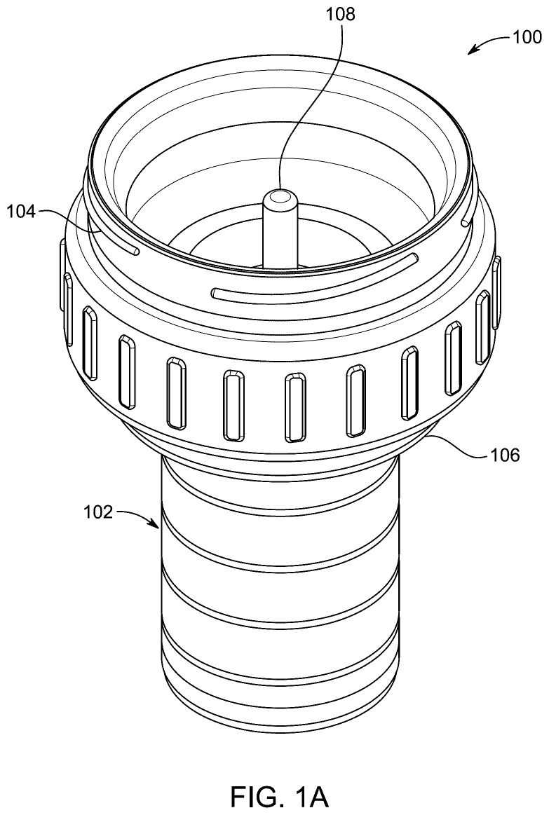

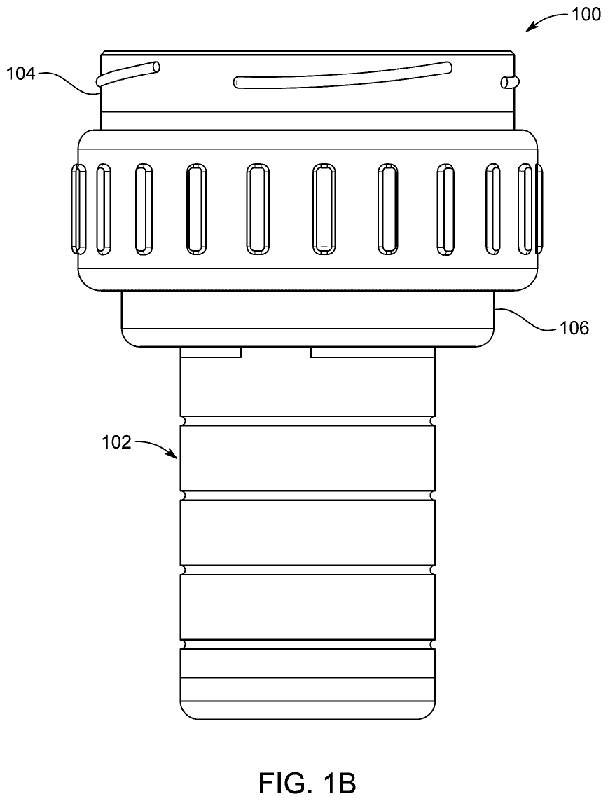

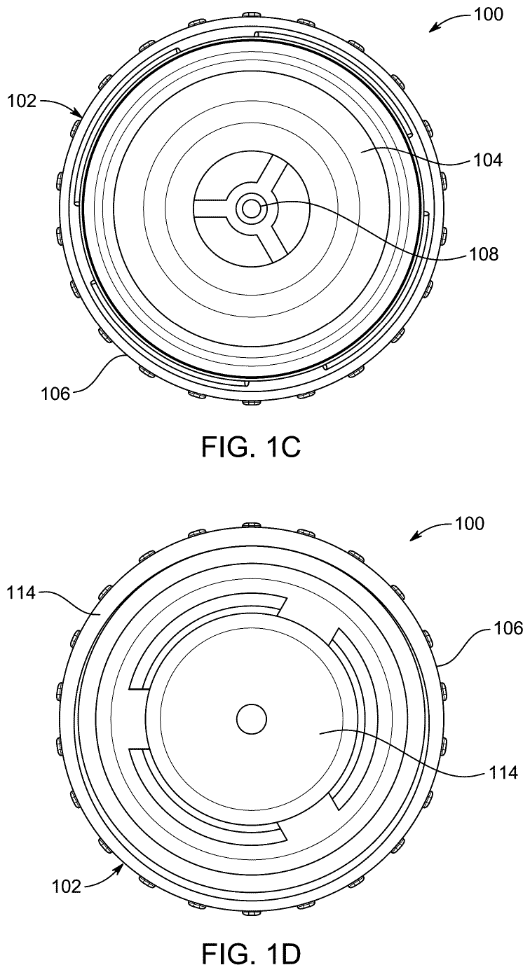

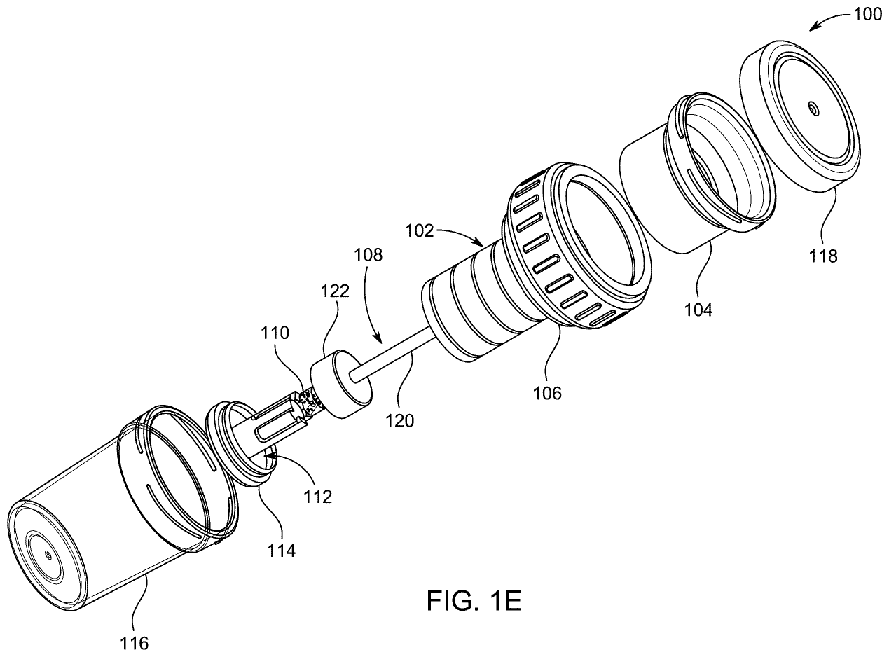

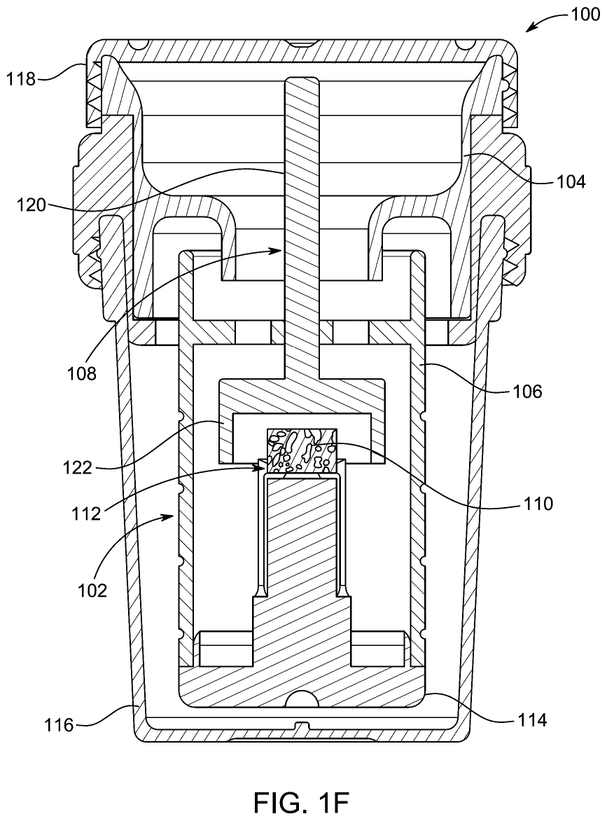

Referring to FIGS. 1A-1F in combination, illustrated are various views of a sample collection device (as represented by reference numeral 100) for the collection of a urine sample, according to certain embodiments. The sample collection device 100 of the present disclosure utilizes a multi-component configuration that integrates mechanical and fluid dynamics principles to achieve automated separation of urine streams. The sample collection device 100 operates through a passive mechanism triggered by the natural flow of urine, requiring no external power sources or manual adjustments during collection. The sample collection device 100 is configured to function across various patient conditions, including those with limited dexterity or mobility issues. The sample collection device 100 is designed for single-use application, eliminating the need for cleaning or sterilization between uses while maintaining cost-effectiveness for healthcare providers

As illustrated, the sample collection device 100 includes a main chassis 102, a funnel 104, a forestream isolation tube 106, a plunger 108, a sponge 110, a sponge holder 112, a bottom cap 114, and a collection cup 116. It may be noted that FIGS. 1A-1D depict the sample collection device 100 without the collection cup 116, for clarity. These components of the sample collection device 100 are assembled together to form an integrated device for collecting urine samples, particularly midstream urine samples. The components of the sample collection device 100 are assembled in a manner that prevents leakage of urine during the collection process, maintaining hygiene and ensuring accurate sample collection. The sample collection device 100 is designed to be held by a user during the collection process. The sample collection device 100 is sized appropriately to be held comfortably while still providing sufficient capacity for collecting an adequate urine sample for testing purposes. The materials used for constructing the sample collection device 100 are medical-grade, ensuring safety for patient use and compatibility with laboratory testing procedures.

In the sample collection device 100, the main chassis 102 serves as the primary structural element of the device, providing support and housing for other components. The main chassis 102 is designed with appropriate dimensions to accommodate the forestream isolation tube 106 internally while maintaining sufficient space around it for urine flow. The material of the main chassis 102 is selected for its durability, biocompatibility, and suitability for medical applications. The main chassis 102 includes features that allow for secure attachment of the bottom cap 114 and proper positioning of the forestream isolation tube 106.

Herein, the main chassis 102 has an open top. The open top of the main chassis 102 provides an entry point for the funnel 104 to be coupled with the inner surface of the top portion of the main chassis 102. This coupling creates a secure connection that maintains the proper alignment of components during use. Additionally, the open top of the main chassis 102 serves as the overflow point for the midstream urine once the forestream isolation tube 106 has been sealed by the plunger 108. As the midstream urine accumulates in the main chassis 102 around the exterior of the forestream isolation tube 106, the level rises until it reaches the open top, at which point the urine begins to overflow into the collection cup 116 positioned below. This overflow mechanism is required for directing the midstream portion of the urine sample into the collection cup 116 for laboratory analysis. The open top may include features such as a slight lip or channel to direct the overflowing urine uniformly into the collection cup 116, enhancing the efficiency of the collection process.

The funnel 104 is configured to couple with an inner surface of a top portion of the main chassis 102, creating a secure connection that prevents leakage during use. The coupling mechanism between the funnel 104 and the main chassis 102 may utilize threads, snap-fit features, or other suitable connection methods that ensure a secure fit while allowing for disassembly if needed. The funnel 104 is specifically designed to guide urine into the device with minimal splashing or spillage, enhancing the hygiene of the collection process. The shape of the funnel 104 is optimized to facilitate easy and comfortable use by patients, with a wide opening that narrows toward the bottom to efficiently direct urine flow.

The forestream isolation tube 106 is responsible for separating the forestream portion of the urine sample in the sample collection device 100. The forestream isolation tube 106 is positioned centrally within the main chassis 102, aligned with a narrow end of the funnel 104 to receive the initial urine flow. The forestream isolation tube 106 has at least one opening at a top portion thereof (as may be seen in FIGS. 1E and 1F). The at least one opening at the top portion of the forestream isolation tube 106 is sized appropriately to allow urine to flow into the tube while maintaining structural integrity. This opening may be circular, oval, or another suitable shape that facilitates efficient flow while being compatible with the sealing action of the plunger 108. The forestream isolation tube 106 extends downward from its top opening, creating a reservoir for collecting the forestream portion of the urine sample. The walls of the forestream isolation tube 106 are of sufficient thickness to maintain structural integrity during use, preventing deformation that could compromise function. The bottom portion of the forestream isolation tube 106 is designed to accommodate the top end of the plunger 108, allowing for a secure fit while still permitting the plunger 108 to move upward when pushed by the expanding sponge 110. The dimensions of the forestream isolation tube 106 are selected to ensure it has sufficient capacity for the forestream portion of the urine sample, with additional capacity to accommodate variation between individuals.

In an embodiment, the forestream isolation tube 106 has a capacity of at least 40 mL. This capacity is specifically selected to ensure that the forestream isolation tube 106 can adequately contain the typical forestream volume of urine, which studies indicate consists of about 5 mL to 20 mL. The minimum capacity of 40 mL provides a safety margin, accommodating variations in forestream volume among different individuals and ensuring that the forestream isolation tube 106 does not overflow before the plunger 108 seals the top opening. The capacity of at least 40 mL is achieved through the dimensions of the forestream isolation tube 106, with its length and diameter calculated to provide the required volume while maintaining a form factor that fits appropriately within the main chassis 102. The substantial capacity of the forestream isolation tube 106 ensures that the device can effectively isolate the contaminated forestream even in cases where the forestream volume is at the higher end of the normal range. This is particularly important for ensuring reliable separation of the forestream from the midstream across a wide range of patients with varying physiological conditions.

Further, in an embodiment, the forestream isolation tube 106 has a diameter smaller than the diameter of the main chassis 102. This dimensional relationship allows for the proper functioning of the sample collection device 100. The smaller diameter of the forestream isolation tube 106 relative to the main chassis 102 creates an annular space between these components. This space allows urine to flow around the outside of the forestream isolation tube 106 and into the collection cup 116 once the forestream isolation tube 106 has been sealed by the plunger 108. The difference in diameters is calculated to provide sufficient flow capacity for the midstream urine while maintaining the structural integrity of both components. This design feature also ensures that once the forestream isolation tube 106 is sealed, the subsequent midstream urine can still flow through the device and into the collection cup 116 without obstruction. The smaller diameter of the forestream isolation tube 106 further allows it to be positioned centrally within the main chassis 102, creating a balanced and stable configuration that enhances the overall performance of the sample collection device 100.

The collection cup 116 having a top opening and the bottom cap 114 therein, as shown in FIGS. 1E and 1F. The collection cup 116 (sometimes also referred to as “urine collection cup” without any limitations) is a standard component used in medical settings for urine collection, making the sample collection device 100 compatible with existing hospital supplies. The collection cup 116 typically has a cylindrical shape with a flat bottom, providing stability when placed on a surface. The top opening of the collection cup 116 is sized appropriately to fit with the bottom portion of the main chassis 102, creating a secure connection that prevents leakage during the collection process. Further, the bottom cap 114 is positioned within the top opening of the collection cup 116, forming part of the interface between the collection cup 116 and the main chassis 102. The capacity of the collection cup 116 is sufficient to contain a standard volume required for laboratory testing, typically around 30-60 mL. The collection cup 116 is generally manufactured from transparent or translucent materials, allowing visual confirmation of the sample volume and characteristics.

In the sample collection device 100, the collection cup 116 is configured to hold a clean sample of urine. The collection cup 116 is specifically positioned and configured to receive only the midstream portion of the urine sample, after the contaminated forestream has been isolated in the forestream isolation tube 106. The term “clean sample” refers to the midstream urine, which typically contains fewer contaminants and is more representative of the conditions within the bladder than the forestream. The materials used for the collection cup 116 are compatible with urine and do not introduce any substances that could affect laboratory results. The design of the collection cup 116 includes features that prevent leakage during and after collection, maintaining the integrity of the sample until it can be tested. The collection cup 116 is also designed to be easily handled by healthcare professionals and laboratory staff, with dimensions and features that facilitate safe transport and processing of the sample.

In an embodiment, the collection cup 116 has a cap 118 (as shown in FIGS. 1E and 1F) configured to couple with the funnel 104 to close a mouth of the funnel 104. The cap 118 is typically the same cap that is ordinarily used with the collection cup 116 in standard medical settings, maintaining compatibility with existing hospital supplies. Before use of the sample collection device 100, this cap is removed from the collection cup 116 and set aside. After the collection process is complete and the collection cup 116 contains the midstream urine sample, the cap 118 is taken and coupled with the funnel 104 at the top of the sample collection device 100. The cap 118 is designed to securely close the mouth of the funnel 104, preventing spillage of any residual urine that may remain in the sample collection device 100. The coupling between the cap 118 and the funnel 104 may utilize threads, snap-fit features, or other suitable connection methods that ensure a secure fit. Once the cap 118 is in place, the sample collection device 100 can be safely handled and disposed of without risk of contamination from the collected forestream urine.

Herein, the collection cup 116 threads onto an inferior aspect of the sample collection device 100. This threaded connection provides a secure attachment between the collection cup 116 and the rest of the sample collection device 100, preventing leakage during the collection process. The threading is designed to be compatible with standard urine collection cups used in medical settings, allowing the sample collection device 100 to work with existing supplies rather than requiring proprietary cups. The threaded connection typically involves corresponding threads on the exterior of the bottom portion of the main chassis 102 or bottom cap 114 and the interior of the top opening of the collection cup 116. The threaded connection allows for easy attachment and detachment of the collection cup 116, facilitating assembly before use and separation after collection for laboratory processing of the midstream sample.

In present embodiments, the urine collection cup 116 is fully enclosed. This design feature ensures that once the midstream urine sample has been collected, it remains contained within a sealed environment, protected from external contamination. When attached to the sample collection device 100, the open top of the urine collection cup 116 connects securely with the main chassis 102 or the bottom cap 114, creating a sealed interface that prevents leakage. After the collection process is complete and the midstream sample has been collected, the urine collection cup 116 can be detached from the rest of the sample collection device 100. Once detached, the standard cap can be screwed onto the urine collection cup 116, fully enclosing the midstream urine sample for transport to the laboratory. Such fully enclosed design of the urine collection cup 116 helps in maintaining the integrity of the urine sample during handling, storage, and transport.

In the sample collection device 100, the bottom cap 114 comprises the sponge holder 112 (as shown in FIGS. 1E and 1F). Specifically, the bottom cap 114 includes the sponge holder 112, which contains the sponge 110 in the assembled device. The bottom cap 114 serves as the interface between the main chassis 102 and the collection cup 116, providing a secure connection while also housing the sponge holder 112. The bottom cap 114 is designed to fit securely with the bottom portion of the main chassis 102, creating a sealed environment that prevents leakage during the collection process. The connection between the bottom cap 114 and the main chassis 102 may utilize threads, snap-fit features, or other suitable connection methods that ensure a secure fit while allowing for disassembly if needed.

In the present configuration, the sponge 110 is contained within the sponge holder 112. The sponge holder 112 is typically a recessed area or compartment within the bottom cap 114, sized appropriately to hold the sponge 110 in its dry, compressed state. The sponge holder 112 includes features that prevent the sponge 110 from being displaced during assembly or use, ensuring that it remains in the proper position to interact with the bottom end of the plunger 108. The sponge holder 112 also includes openings or channels that allow urine to reach the sponge 110, triggering its expansion during the collection process. The bottom cap 114 and sponge holder 112 are manufactured from materials that are compatible with the sponge 110 and do not interfere with its expansion properties.

In its dry, compressed state, the sponge 110 is contained within the sponge holder 112, positioned directly below the bottom end of the plunger 108. This positioning ensures that when the sponge 110 expands, it exerts force directly on the plunger 108, maximizing the efficiency of the sealing mechanism. The sponge 110 is sized appropriately to fit within the sponge holder 112 in its compressed state while having sufficient material to achieve the necessary expansion for pushing the plunger 108. The expansion characteristics of the sponge 110 ensures that the forestream isolation tube 106 is sealed at the appropriate point in the collection process.

The sponge 110 is specifically selected for its ability to rapidly absorb liquid and expand significantly upon contact with urine. In an embodiment, the sponge 110 is a cellulose sponge (with the two terms being interchangeably used hereinafter, without any limitations). The cellulose sponge 110 is particularly suitable for this application due to its high absorption properties and substantial expansion capability. The cellulose sponge can expand to more than 10× of its original size (when it absorbs urine). This provides sufficient force to push the plunger 108 upward and seal the forestream isolation tube 106. Further, the absorption rate of cellulose sponge is rapid, ensuring that the sealing mechanism is triggered promptly when the forestream urine reaches the sponge 110. This rapid response facilitates the proper timing of the separation between forestream and midstream portions of the urine sample. The material properties of the cellulose sponge are selected to ensure rapid absorption and expansion upon contact with urine, while being compatible with the medical nature of the sample collection device 100.

The sponge 110 is a key element in the automatic operation of the sample collection device 100. When dry, the sponge 110 is in a compressed state, occupying minimal space within the sponge holder 112. Upon contact with urine, the sponge 110 rapidly absorbs the liquid and undergoes an expansion, increasing to more than 10 times its original volume. This expansion generates significant mechanical force, which is directed upward against the bottom end of the plunger 108. The force generated by the expanding sponge 110 is sufficient to overcome any resistance and push the plunger 108 upward to seal the opening of the top portion of the forestream isolation tube 106. The rapid expansion rate of the sponge 110 ensures that the sealing occurs promptly after the forestream has been collected, optimizing the separation between forestream and midstream portions of the urine sample. Further, the substantial expansion ensures that the seal remains in place throughout the collection process, preventing mixing of the forestream and midstream portions.

The plunger 108 has a top end inserted in a bottom portion of the forestream isolation tube 106 and a bottom end extending downward proximal to and/or contacting the sponge 110 (as may be seen in FIGS. 1E and 1F). This positioning of the plunger 108 allows for the automatic sealing mechanism that separates the forestream and midstream portions of the urine sample. The top end of the plunger 108 is designed to fit within the bottom portion of the forestream isolation tube 106 while still allowing for vertical movement. The fit between the top end of the plunger 108 and the forestream isolation tube 106 is designed to maintain proper alignment during the upward movement of the plunger 108, ensuring that the plunger 108 can effectively seal the opening at the top portion of the forestream isolation tube 106 when pushed upward. The bottom end of the plunger 108 extends downward toward the sponge 110, positioned either in close proximity to or directly contacting the sponge 110 in its dry, compressed state. This proximity or contact ensures that the force generated by the expanding sponge 110 is efficiently transferred to the plunger 108, initiating upward movement with minimal delay.

In present embodiments, the plunger 108 has a cylinder portion 120. The cylinder portion 120 forms the main body of the plunger 108, with dimensions to fit within the bottom portion of the forestream isolation tube 106 while allowing for vertical movement. The length of the cylinder portion 120 is determined by the distance between the bottom portion of the forestream isolation tube 106 and the top opening that needs to be sealed, ensuring that the plunger 108 can effectively close the opening when pushed upward. The diameter of the cylinder portion 120 is smaller than the inner diameter of the forestream isolation tube 106, allowing for movement while minimizing lateral play that could compromise the sealing action.

The plunger 108 also has a base 122 having a diameter larger than a diameter of the cylinder portion 120. The larger diameter of the base 122 provides a greater surface area for contact with the expanding sponge 110, allowing the force of expansion to be effectively transferred to the plunger 108. This increased contact area ensures that even if the expansion of the sponge 110 is uneven, sufficient force is still applied to the plunger 108 to achieve the desired upward movement. The larger diameter of the base 122 also prevents the plunger 108 from being pushed too far into the forestream isolation tube 106, as the base 122 cannot pass through the narrower bottom opening of the forestream isolation tube 106, acting as a mechanical stop to ensure proper positioning of the plunger 108 during use.

The sample collection device 100 has the cap 118 that is used to seal the sample collection device 100 when it is attached to the urine collection cup 116. This cap 118 is typically the same cap that is normally used with the collection cup 116 (as discussed in the preceding paragraphs) in standard medical settings. After the collection process is complete, the cap 118 is coupled with the funnel 104 at the top of the sample collection device 100, sealing the entire assembly. This sealing action prevents spillage of any urine contained within the device, including both the forestream in the forestream isolation tube 106 and any residual midstream in the main chassis 102. Herein, the cap 118 is removed before collection of the urine sample. That is, before the sample collection device 100 is used, the cap 118 is unscrewed from the collection cup 116 and set aside for later use. The removal of the cap 118 opens the system, allowing urine to enter the funnel 104 during the collection process. After the collection process is complete, the same cap 118 can be used to seal the sample collection device 100 by attaching it to the funnel 104, creating a closed system for safe handling and disposal.

The sample collection device 100 is fluidly configured such that the urine sample directed into the funnel 104 flows through an inner compartment of the funnel 104, into the main chassis 102, and through the opening of the top portion of the forestream isolation tube 106 to the sponge 110. That is, when a patient begins urination, the initial stream is directed into the funnel 104, which guides the urine through its inner compartment and into the main chassis 102. The main chassis 102 temporarily holds the urine, directing it toward the opening at the top portion of the forestream isolation tube 106. The forestream portion of the urine, which typically contains the highest concentration of contaminants, flows through this opening and into the forestream isolation tube 106. The forestream isolation tube 106 channels the urine downward toward the bottom of the sample collection device 100, where it contacts the sponge 110 contained in the sponge holder 112. The fluid path is designed to minimize turbulence and splashing, ensuring smooth flow of urine through the sample collection device 100.

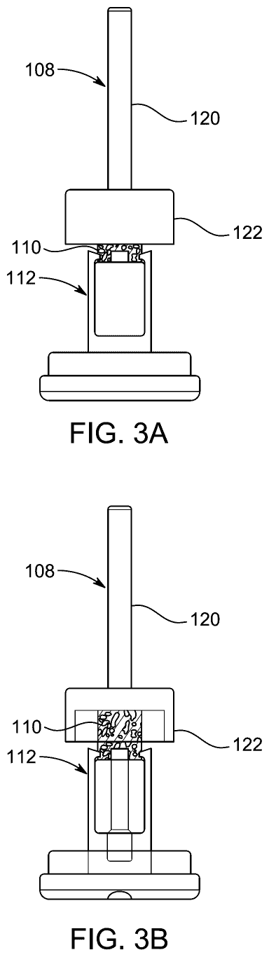

As the urine sample travels to the sponge 110, the sponge 110 absorbs the urine sample and expands, pushing the base 122 of the plunger 108 upwards to seal the opening of the top portion of the forestream isolation tube 106 (as depicted in FIGS. 3A and 3B). This sequence represents the automatic operation of the sample collection device 100 that enables separation of the forestream and midstream portions of the urine sample. When the forestream urine reaches the sponge 110, the cellulose material rapidly absorbs the liquid, triggering an expansion of the sponge 110 to more than 10 times its original size. This expansion generates significant mechanical force directed upward against the base 122 of the plunger 108. The force exerted by the expanding sponge 110 overcomes any resistance to movement, pushing the plunger 108 upward within the forestream isolation tube 106. As the plunger 108 moves upward, its top end eventually reaches the opening at the top portion of the forestream isolation tube 106. When the top end of the plunger 108 contacts this opening, it forms a seal that prevents any further urine from entering the forestream isolation tube 106. The timing of this sealing action is determined by the rate of urine flow and the absorption and expansion characteristics of the sponge 110, typically occurring after approximately 5-20 mL of forestream urine has been collected in the forestream isolation tube 106. The seal formed by the plunger 108 is secure and reliable, ensuring complete separation of the forestream from the subsequent midstream urine.

When the opening of the top portion of the forestream isolation tube 106 is sealed, the urine sample enters the main chassis 102, fills the main chassis 102, and overflows through the open top of the main chassis 102 into the collection cup 116. This represents the second phase of the collection process, where the midstream portion of the urine sample is captured for analysis. Herein, once the plunger 108 has sealed the forestream isolation tube 106, the subsequent urine flow (midstream) can no longer enter the forestream isolation tube 106. Instead, the midstream urine accumulates in the main chassis 102 around the exterior of the forestream isolation tube 106. As the patient continues to urinate, the level of urine in the main chassis 102 rises until it reaches the open top of the main chassis 102. At this point, the urine begins to overflow through this opening and into the collection cup 116 positioned below. The collection cup 116 is specifically designed to hold this clean midstream sample, which is typically less contaminated than the forestream and therefore more suitable for accurate laboratory analysis. The overflow mechanism ensures that the collection cup 116 receives only the midstream portion of the urine sample, after the forestream has been isolated in the forestream isolation tube 106. The capacity of the main chassis 102 and the collection cup 116 are appropriately sized to accommodate typical midstream volumes, ensuring that sufficient sample is collected for standard laboratory tests without overflow of the collection cup 116.

A midstream portion of the urine is diverted into the main chassis 102 and the collection cup 116 when the cellulose sponge 110 expands and closes the plunger 108. This diversion mechanism enables the automatic separation of the forestream and midstream portions of the urine sample. As discussed, initially, the forestream portion of the urine enters the forestream isolation tube 106 through the opening at its top portion. When the forestream urine reaches the sponge 110, it triggers the rapid expansion of the cellulose sponge 110, which pushes the plunger 108 upward to seal the opening of the forestream isolation tube 106. Once this seal is established, subsequent urine flow (the midstream portion) can no longer enter the forestream isolation tube 106. Instead, the midstream urine accumulates in the main chassis 102 around the exterior of the forestream isolation tube 106. As the level of midstream urine in the main chassis 102 rises, it eventually overflows through the open top of the main chassis 102 and into the collection cup 116 positioned below. This diversion mechanism operates automatically without requiring any manual intervention by the patient or healthcare provider, enhancing the reliability and consistency of the midstream collection process. The timing of the diversion is determined by the absorption and expansion properties of the cellulose sponge 110, ensuring that the forestream is consistently isolated before the midstream is collected, regardless of variations in urine flow rate or volume.

In present embodiments, the components of the sample collection device 100 are an integrally-connected unit. That is, when assembled, the various components of the sample collection device 100 form a cohesive and functionally integrated system, with each part positioned relative to the others to ensure proper operation. The main chassis 102, the funnel 104, the forestream isolation tube 106, the plunger 108, the sponge 110, the sponge holder 112, the bottom cap 114, and the collection cup 116 all incorporate specific design elements that facilitate proper interconnection. These may include alignment guides, snap-fit features, threaded connections, or other joining mechanisms that ensure each component is securely attached in the correct position. The integral connection of components enhances the reliability of the sample collection device 100, minimizing the risk of improper assembly that could compromise function. It may be appreciated that the said integrally-connected nature does not preclude the attachment or detachment of certain components, such as the collection cup 116 or cap, which are designed to be removed as part of the normal operation of the sample collection device 100.

Further, the sample collection device 100 components are fully enclosed unless the cap 118 is removed. That is, when fully assembled with the cap 118 in place, all internal components of the sample collection device 100 are contained within a sealed environment. The cap, which couples with the funnel 104 at the top of the sample collection device 100, serves as the primary closure point. When the cap 118 is in place, the entire system is sealed, containing any urine within the device and preventing external contaminants from entering. This fully enclosed nature is important for maintaining the cleanliness of the sample collection device 100 before use and for containing any collected urine after use. The enclosure of components also facilitates safe handling and disposal of the device after collection, minimizing the risk of contamination to healthcare providers or the environment.

Herein, both the forestream portion and the midstream portion remain enclosed within the sample collection device 100 during the urine collection process. This complete containment of both urine portions enhances hygiene and reducing the risk of contamination during collection. The forestream portion is contained within the forestream isolation tube 106. Once the forestream has been collected and the plunger 108 has sealed the forestream isolation tube 106, this portion of the urine remains fully contained within the sample collection device 100. The midstream portion initially accumulates in the main chassis 102 and then overflows into the collection cup 116. Throughout the entire collection process, neither the forestream nor the midstream is exposed to the external environment, except at the point of entry through the funnel 104. This enclosed collection process contrasts with traditional mid-stream collection methods, which typically involve diverting the forestream into the toilet before redirecting the midstream into a collection cup, a process that is messy, difficult to control, and prone to contamination. The complete containment of both urine portions within the sample collection device 100 enhances the hygiene of the collection process, reduces the risk of sample contamination, and simplifies the procedure for patients, potentially increasing compliance with proper collection techniques.

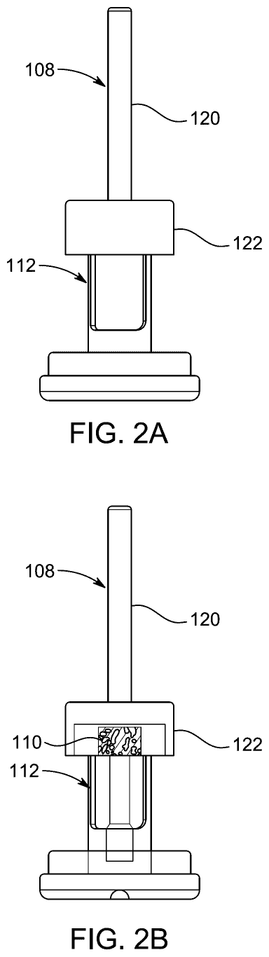

Referring to FIGS. 2A and 2B in combination, illustrated are views of the plunger 108 showing its movement before expansion of the sponge 110 therein, according to certain embodiments. As discussed, the plunger 108 is responsible for sealing the forestream isolation tube 106 once a sufficient amount of forestream urine has been collected in the sample collection device 100. The plunger 108 comprises the cylinder portion 120 and the base 122. The cylinder portion 120 is designed to fit within the bottom portion of the forestream isolation tube 106, while still allowing for vertical movement. The base 122 has a diameter larger than the diameter of the cylinder portion 120, providing a larger surface area for contact with the expanding sponge 110. This design ensures that when the sponge 110 expands upon contact with urine, it exerts sufficient force on the base 122 to push the entire plunger 108 upward. The surface of the plunger 108, particularly the top end that seals against the forestream isolation tube 106, is designed to create an effective seal when pushed into position.

Referring to FIGS. 3A and 3B in combination, illustrated are views of the plunger 108 showing its movement after expansion of the sponge 110 therein, according to certain embodiments. This demonstrates the operation of the plunger 108 once the sponge 110 has expanded. When the forestream urine comes into contact with the sponge 110, the sponge 110 rapidly absorbs the liquid and expands significantly. This expansion generates an upward force on the base 122 of the plunger 108, causing the plunger 108 to move upward within the forestream isolation tube 106. As the plunger 108 moves upward, the cylinder portion 120 slides along the inner surface of the forestream isolation tube 106 until the top end of the plunger 108 reaches the opening at the top portion of the forestream isolation tube 106. The upward movement of the plunger 108 is entirely driven by the expansion of the sponge 110, requiring no manual intervention or external energy source. When the top end of the plunger 108 contacts this opening, it forms a seal that prevents any further urine from entering the forestream isolation tube 106. This sealing action effectively isolates the forestream portion of the urine that has already been collected within the forestream isolation tube 106, ensuring that it does not mix with the subsequently collected midstream portion. The seal created by the plunger 108 is secure and reliable, maintaining separation between the forestream and midstream portions throughout the remainder of the collection process.

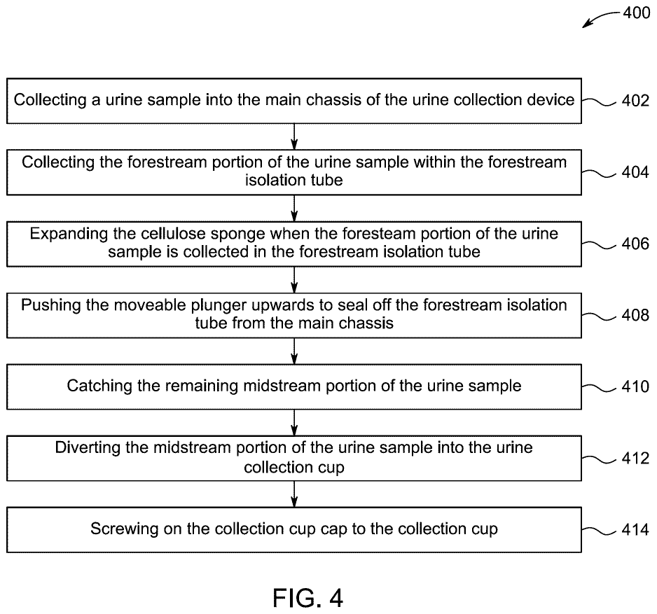

Referring now to FIG. 4, a flowchart of a method (as represented by reference numeral 400) for collecting the midstream portion of the urine sample is illustrated. The method 400 includes a series of steps. These steps are only illustrative, and other alternatives may be considered where one or more steps are added, one or more steps are removed, or one or more steps are provided in a different sequence without departing from the scope of the present disclosure. Various variants described above, with respect to the aforementioned sample collection device 100 apply mutatis mutandis to the present method 400.

At step 402, the method 400 includes collecting a urine sample into the main chassis 102 of the urine collection device 100. This initial step involves the patient directing their urine stream into the funnel 104 at the top of the device. The funnel 104 guides the urine into the main chassis 102, where it then flows toward the opening at the top portion of the forestream isolation tube 106. The collection of the urine sample is a continuous process that begins with the start of urination and continues until a sufficient sample has been obtained. The funnel 104 is designed to minimize splashing and spillage during collection, enhancing the hygiene of the process. The main chassis 102 temporarily holds the urine as it is directed toward the appropriate pathways within the device. The patient does not need to manually redirect their urine stream during collection, as the device automatically manages the separation of forestream and midstream portions.

At step 404, the method 400 includes collecting the forestream portion of the urine sample within the forestream isolation tube 106. As the initial urine stream enters the main chassis 102, it is directed toward the opening at the top portion of the forestream isolation tube 106. The forestream, which typically consists of the first 5-20 mL of urine, flows through this opening and into the forestream isolation tube 106. The forestream isolation tube 106 has a capacity of at least 40 mL, ensuring that it can contain the entire forestream portion without overflow. The forestream is typically considered to have a higher concentration of contaminants than the midstream, making its isolation desired for obtaining a clean midstream sample for accurate laboratory analysis. The collection of the forestream within the forestream isolation tube 106 happens automatically as part of the normal urine flow through the sample collection device 100, requiring no special action or attention from the patient.

At step 406, the method 400 includes expanding the cellulose sponge 110 when the forestream portion of the urine sample is collected in the forestream isolation tube 106. As the forestream urine flows through the forestream isolation tube 106, it eventually reaches the bottom of the tube where it contacts the sponge 110 contained within the sponge holder 112. Upon contact with urine, the sponge 110 begins to rapidly absorb the liquid, triggering its expansion. The sponge 110, being formed of cellulose, is capable of expanding to more than 10 times its original size when it absorbs urine. The expansion of the sponge 110 is a physical reaction to liquid absorption and occurs without requiring any energy input or manual activation. The rate of expansion is rapid enough to ensure timely activation of the sealing mechanism, while still allowing sufficient forestream to be collected before the seal is established.

At step 408, the method 400 includes pushing the moveable plunger 108 upwards to seal off the forestream isolation tube 106 from the main chassis 102. As the sponge 110 expands upon contact with the forestream urine, it exerts an upward force on the base 122 of the plunger 108. This force causes the plunger 108 to move upward within the forestream isolation tube 106, with the cylinder portion 120 of the plunger 108 sliding along the inner surface of the forestream isolation tube 106. As the plunger 108 moves upward, its top end eventually reaches the opening at the top portion of the forestream isolation tube 106. When the top end of the plunger 108 contacts this opening, it forms a seal that prevents any further urine from entering the forestream isolation tube 106. The seal effectively isolates the forestream urine that has already been collected within the forestream isolation tube 106, separating it from the subsequent midstream flow.

At step 410, the method 400 includes catching the remaining midstream portion of the urine sample. Once the plunger 108 has sealed the opening of the forestream isolation tube 106, the subsequent urine flow (midstream) can no longer enter the forestream isolation tube 106. Instead, the midstream urine accumulates in the main chassis 102 around the exterior of the forestream isolation tube 106. As the level of midstream urine in the main chassis 102 rises, it eventually reaches the open top of the main chassis 102. The midstream portion of the urine is typically less contaminated than the forestream and is therefore more suitable for laboratory analysis. The catching of the midstream happens automatically as part of the normal operation of the sample collection device 100, requiring no manual intervention from the patient. The timing of the transition from forestream to midstream collection is determined by the expansion rate of the sponge 110 and the resulting sealing action of the plunger 108, typically occurring after approximately 5-20 mL of forestream has been collected.

At step 412, the method 400 includes diverting the midstream portion of the urine sample into the urine collection cup 116. As the midstream urine accumulates in the main chassis 102 and reaches the open top, it begins to overflow through this opening and into the collection cup 116 positioned below. This diversion happens automatically as a consequence of the sealed forestream isolation tube 106 and the continued flow of urine into the device. The path of the midstream urine from the main chassis 102 to the collection cup 116 is designed to minimize turbulence and splashing, ensuring smooth flow into the collection cup 116. The collection cup 116 is specifically positioned to receive the overflowing midstream urine, with its top opening aligned with the overflow point of the main chassis 102. The diversion mechanism ensures that only the midstream portion enters the collection cup 116, maintaining the separation between forestream and midstream that is essential for accurate laboratory analysis.

At step 414, the method 400 includes screwing on the collection cup cap 118 to the collection cup 116. After the urine collection is complete and a sufficient midstream sample has been collected in the collection cup 116, the patient or healthcare provider retrieves the cap 118 that was removed before collection. The cap 118 is then screwed onto the top of the funnel 104, creating a sealed system that contains both the forestream in the forestream isolation tube 106 and any residual midstream in the main chassis 102. Once the cap 118 is securely attached, the entire sample collection device 100, including the collection cup 116 containing the midstream sample, can be safely handled and transported to the laboratory for analysis.

The sample collection device 100 and the method 400 of the present disclosure offer advantages over existing collection devices and traditional methods by addressing multiple limitations that have hindered effective urine sample collection. The automatic separation of the forestream and midstream portions eliminates the need for patients to manually redirect their urine stream during collection, a process that is prone to error and often leads to sample contamination. The sample collection device 100 maintains compatibility with standard collection cups and caps used in medical settings, allowing for easy integration into existing healthcare workflows without requiring proprietary components. The forestream isolation tube 106 ensures adequate containment of the forestream portion, accommodating variations in forestream volume among different individuals. The plunger 108 provides mechanical advantage for the sealing mechanism, ensuring reliable operation across various conditions. The sponge 110 generates sufficient force to activate the sealing mechanism without requiring external power sources or manual triggers. The fully enclosed nature of the sample collection device 100 components, unless the cap 118 is removed, maintains a closed system that prevents leakage or contamination throughout the collection process. These features collectively result in improved sample quality, increased patient compliance, and potential reduction in unnecessary antibiotic prescriptions due to more accurate laboratory results.

While specific embodiments of the invention have been described, it should be understood that various modifications and alternatives may be implemented without departing from the spirit and scope of the invention. The above-described hardware description is a non-limiting example of corresponding structure for performing the functionality described herein. Numerous modifications and variations of the present disclosure are possible in light of the above teachings. It is therefore to be understood that within the scope of the appended claims, the invention may be practiced otherwise than as specifically described herein.

1. A sample collection device for the collection of a urine sample, comprising:
a main chassis and a funnel therein, the funnel configured to couple with an inner surface of a top portion of the main chassis;
a forestream isolation tube having at least one opening at a top portion of the forestream isolation tube;
a collection cup having a top opening and a bottom cap therein, the bottom cap comprising a sponge holder;
a sponge contained within the sponge holder; and
a plunger with a base having a top end inserted in a bottom portion of the forestream isolation tube and a bottom end extending downward proximal to and/or contacting the sponge,
wherein the sample collection device is fluidly configured such that the urine sample directed into the funnel flows through an inner compartment of the funnel, into the main chassis, and through the opening of the top portion of the forestream isolation tube to the sponge, and wherein as the urine sample travels to the sponge, the sponge absorbs the urine sample and expands, pushing the base of the plunger upwards to seal the opening of the top portion of the forestream isolation tube.

2. The sample collection device of claim 1, wherein the main chassis has an open top.

3. The sample collection device of claim 1, wherein the forestream isolation tube has a capacity of at least 40 mL.

4. The sample collection device of claim 1, wherein the collection cup is configured to hold a clean sample of urine.

5. The sample collection device of claim 1, wherein the collection cup has a cap configured to couple with the funnel to close a mouth of the funnel.

6. The sample collection device of claim 1, wherein the forestream isolation tube has a diameter smaller than the diameter of the main chassis.

7. The sample collection device of claim 1, wherein the plunger has a cylinder portion.

8. The sample collection device of claim 7, wherein the base of the plunger having a diameter larger than a diameter of the cylinder portion.

9. The sample collection device of claim 1, wherein when the opening of the top portion of the forestream isolation tube is sealed, the urine sample enters the main chassis, fills the main chassis, and overflows through the open top of the main chassis into the collection cup.

10. The sample collection device of claim 1, wherein the sponge is a cellulose sponge.

11. The sample collection device of claim 1, wherein the collection cup threads onto an inferior aspect of the sample collection device.

12. The sample collection device of claim 1, wherein a midstream portion of the urine is diverted into the main chassis and the collection cup when the cellulose sponge expands and closes the plunger.

13. The sample collection device of claim 10, wherein the cellulose sponge can expand to more than 10× of its original size.

14. The sample collection device of claim 1, wherein the sample collection device has a cap that is used to seal the sample collection device when it is attached to the urine collection cup.

15. The sample collection device of claim 14, wherein the cap is removed before collection of the urine sample.

16. The sample collection device of claim 1, wherein the main chassis, the forestream isolation tube, the collection cup, the sponge, and the plunger are an integrally-connected unit.

17. The sample collection device of claim 1, wherein the urine collection cup is fully enclosed.

18. The sample collection device of claim 1, wherein the sample collection components are fully enclosed unless the cap is removed.

19. The sample collection device of claim 1, wherein both the forestream portion and the midstream portion remain enclosed within the sample collection device during the urine collection process.

20. A method for collecting the midstream portion of the urine sample, comprising
collecting a urine sample into the main chassis of the urine collection device;
collecting the forestream portion of the urine sample within the forestream isolation tube;
expanding the cellulose sponge when the forestream portion of the urine sample is collected in the forestream isolation tube;
pushing the plunger upwards to seal off the forestream isolation tube from the main chassis;
catching the remaining midstream portion of the urine sample;
diverting the midstream portion of the urine sample into the urine collection cup; and
screwing on the collection cup cap to the collection cup.
